(19) United States
(12) Patent Application Publication
(10) Pub. No.: US 2026/0363968 A1
(43) Pub. Date: Nov. 19, 2026

(54) COMPOSITION COMPRISING AEROGELS BASED ON BIOPOLYMERS

(71) Applicant: aerogel-it GmbH, Osnabrueck (DE)

(72) Inventors: Marc Fricke, Osnabrueck (DE); Raman Subrahmanyam, Osnabrueck (DE); Dirk Weinrich, Osnabrueck (DE)

(73) Assignee: aerogel-it GmbH, Osnabrueck (DE)

(21) Appl. No.: 19/514,964

(22) PCT Filed: Sep. 6, 2024
(86) PCT No.: PCT/EP2024/074950

§ 371 Date: Feb. 27, 2026

(30) Foreign Application Priority Data
Sep. 8, 2023 (EP) 23196292.9

(51) Int. Cl. A61K 8/04; A61K 8/73; A61K 8/81; B01J 13/00

(52) U.S. Cl. CPC A61K 8/042; A61K 8/73; A61K 8/731; A61K 8/733; A61K 8/8147; B01J 13/0065

(57) ABSTRACT

The present invention relates to a composition in the form of a organogel, emulsion or bigel, comprising at least one non-aqueous liquid or pasty phase, and an aerogel powder added to the at least one non-aqueous phase based on at least one polyionic biopolymer, where the viscosity of the composition is greater than the viscosity of the at least one non-aqueous phase, and wherein the aerogel powder has a density of 30 g/l or less and where the particle size of the aerogel powder is in the range of 0.5 to 250 micrometers. The present invention also relates to a method for preparing a composition, comprising mixing non-aqueous ingredients comprising at least one non-aqueous liquid or pasty component, and at least one aerogel powder based on at least one polyionic biopolymer, where the viscosity of the composition is greater than the viscosity of the at least one non-aqueous phase, and wherein the aerogel powder has a density of 30 g/l or less and a particle size in the range of 0.5 to 250 micrometers. Furthermore, the present invention relates to the use of a composition according to the invention or a composition obtained or obtainable by the process according to the present invention for food applications, for cosmetic applications, for biomedical applications, for agricultural applications, for consumer applications, for construction applications, for adhesive applications, for coating applications, for paint applications, for fragrance release applications, or for pharmaceutical applications.

The present invention relates to a composition in the form of an organogel, emulsion or bigel, comprising at least one non-aqueous liquid or pasty phase, and an aerogel powder added to the at least one non-aqueous phase based on at least one polyionic biopolymer, where the viscosity of the composition is greater than the viscosity of the at least one non-aqueous phase, and wherein the aerogel powder has a density of 30 g/l or less and where the particle size of the aerogel powder is in the range of 0.5 to 250 micrometers. The present invention also relates to a method for preparing a composition, comprising mixing non-aqueous ingredients comprising at least one non-aqueous component, and at least one aerogel powder based on at least one polyionic biopolymer, where the viscosity of the composition is greater than the viscosity of the at least one non-aqueous phase, and wherein the aerogel powder has a density of 30 g/l or less and a particle size in the range of 0.5 to 250 micrometers. Furthermore, the present invention relates to the use of a composition according to the invention or a composition obtained or obtainable by the process according to the present invention for food applications, for cosmetic applications, for biomedical applications, for agricultural applications, for consumer applications, for construction applications, for adhesive applications, for coating applications, for paint applications, for fragrance release applications, or for pharmaceutical applications.

Conventionally, a cosmetic composition formulator uses emulsified systems combining an aqueous phase for freshness and an oily phase for comfort. The strong point of these systems is also that they allow the combination, within the same composition, of cosmetic ingredients or active agents that have different affinities with respect to these two aqueous and oily phases. Unfortunately, for a given emulsified system, it is often necessary to adjust the composition in case different additives are added. To avoid these problems, compounds which induce gelling such as for example aerogels are used in cosmetic compositions. Also, the use of structuring polymers or cellulose is described in the state of the art. For example, WO 2012/084780A2 discloses a cosmetic composition comprising a mixture of hydrophobic silica aerogel particles and a linear silicone oil.

For several applications it would be preferable to use natural products as additives, in particular in personal care applications.

In principle, aerogels based on biopolymers are known. Also oleogels based on various bioaerogels such as proteins are in principle known and are for example described in “Aerogels as porous structures for food applications: Smart ingredients and novel packaging materials”, Lara Manzocco et al in Food Structure, vol 28, 2021, 100188 or “Iron (iii)-cross-linked alginate hydrogels: a critical review”, Daniel Massana Roquero et al. Mater. Adv., 2022, 3, 1849-1873. Also, PCT/EP2023/055870 discloses aerogels based on bio-based polymers and polyionic biopolymers.

However, often large amounts of the materials are necessary to obtain compositions suitable for personal care applications. It was therefore an object to the present invention to provide compositions suitable for personal care applications comprising low amounts of aerogel. It was also an object of the present invention to provide compositions comprising bio-based materials.

According to the present invention, this object has been solved by a composition in the form of an organogel, emulsion or bigel, comprising
(i) at least one non-aqueous liquid or pasty phase
(ii) an aerogel powder added to the at least one non-aqueous phase based on at least one polyionic biopolymer,
   where the viscosity of the composition is greater than the viscosity of the at least one non-aqueous phase, and wherein the aerogel powder has a density of 30 g/l or less and where the particle size of the aerogel powder is in the range of 0.5 to 250 micrometers.

It has surprisingly been found that the compositions according to the invention can be prepared using only small amounts of aerogel. The aerogel powders used have a low density and at the same time a high absorption rate for non-aqueous liquids. Depending on the composition the aerogel powder may form gels with an additional aqueous phase, the non-aqueous phase or both. Preferably, the powder forms a gel with the non-aqueous phase.

The compositions according to the invention also prove to be very stable. Preferably, the composition according to the present invention is an oleogel, an emulsion or a bigel, more preferably an oleogel. An oleogel can be described as a system in which the process technology changes the physical properties of the oil without having to chemically modify them. A bigel typically results from mixing a hydrogel and an organogel.

The absorption capacity measured at the wet point, noted Wp, corresponds to the amount of oil that needs to be added to 100 g of particles in order to obtain a homogeneous paste. It is measured according to the wet point method or the method for determining the oil uptake of a powder described in standard NF T 30-022. It corresponds to the amount of oil adsorbed onto the available surface of the powder and/or absorbed by the powder by measuring the wet point, described below.

An amount, m=2 g, of powder is placed on a glass plate, and the oil (e.g. isononyl isononanoate) is then added dropwise. After addition of 4 to 5 drops of oil to the powder, mixing is performed using a spatula, and addition of oil is continued until conglomerates of oil and powder have formed. From this point, the oil is added one drop at a time and the mixture is then triturated with the spatula. The addition of oil is stopped when a firm, smooth paste is obtained. This paste must be able to be spread on the glass plate without cracking or forming lumps. The volume Vs (expressed in ml) of oil used is then noted.

The oil uptake corresponds to the ratio Vs/m.

Preferably, the aerogel has an absorption capacity at the wet point of more than 20 ml/g for the non-aqueous phase of the composition to be formed.

Measurement of viscosity is in principle known from the state of the art. For example 150 g of the composition can be measured in a suitable container with a rotary viscosimeter IKA rotavisc me-vi at 50 rpm rotation using a paddle suitable for the viscosity range according to the manufacturer instructions and at a temperature of 23 °C according to DIN 53019.

The composition may contain components that are liquid or pasty at room temperature and may itself be liquid or pasty at room temperature. The composition may also be solid at room temperature such as a lipstick formulation for example if it contains components such as waxes as part of the non-aqueous phase that can be liquefied or melted by heating for combination with the aerogel powder and that solidify upon cooling to room temperature.

The viscosity of the composition during preparation according to the present invention may for example be in a range up to 100000 mPa*s, preferably in a range of from 50 to 10000 mPa*s, in particular in a range of from 200 to 5000 mPa*s.

According to the present invention the non-aqueous phase is liquid or pasty, in particular at room temperature.

The viscosity of the non-aqueous phase can vary as long as the non-aqueous phase can be combined homogeneously with the aerogel. It is also possible to use heating or other suitable methods to adjust the viscosity for a mixing step. Preferably, the non-aqueous phase is liquid or liquefied for facile combination with the aerogel. The addition of the aerogel preferably increases the viscosity of the at least one non-aqueous phase at least by 100%, more preferable at least by 1000%.

Non-aqueous components suitable for the composition according to the present invention are in principle known to the person skilled in the art. In principle, any non-aqueous phase may be used which is liquid or liquefied and forms a composition with the aerogel where the viscosity of the non-aqueous phase is increased, preferably increased by at least 100%. Particularly preferred are non-aqueous phases which form an oleogel with the aerogel.

The viscosity of the non-aqueous liquid or liquefied phase typically is in the range of from 50 to 5000 mPa*s.

The non-aqueous phases may for example contain one or more of esters of saturated or unsaturated, linear or branched C1-C26 aliphatic monoacid or polyacids and of saturated or unsaturated, linear or branched C1-C26 aliphatic monoalcohol or polyalcohol, or silicones, silicon oils or fluorosilicon oils, non-aqueous fragrances, aromas, nutrients, preservatives, pH regulators, film formers, dextrane esters, sucrose esters, moisturizers, synthetic polymers including block copolymers or semi-crystalline polymers, structuring polymers, silicone elastomers, pigments, mixed pigments or pigment composites, opacifiers, blurring fillers, soft-focus fillers, spherical or lamellar inorganic or organic powders, perlite, sun protection agents. The non-aqueous phases may also contain further components.

According to a further embodiment, the present invention is also directed to the composition as disclosed above, wherein the non-aqueous phase contains one or more of esters of saturated or unsaturated, linear or branched C1-C26 aliphatic monoacid or polyacids and of saturated or unsaturated, linear or branched C1-C26 aliphatic monoalcohol or polyalcohol, or silicones, or silicon oils or fluorosilicon oils, non-aqueous fragrances, aromas, nutrients, preservatives, pH regulators, film formers, dextran esters, sucrose esters, moisturizers, synthetic polymers including block copolymers or semi-crystalline polymers, structuring polymers, sun protection agents, silicone elastomers, pigments, mixed pigments or pigment composites, opacifiers, blurring fillers, soft-focus fillers, spherical or lamellar inorganic or organic powders, perlite, sun protection agents.

In the context of the present invention, biopolymers or bio-based polymers are understood to be polymers which are obtained from renewable resources (algae, bacteria, microorganisms, plants, etc.). Bio-based polymers can be obtained mainly by two different ways: the direct production of polymers or the production of bio-based monomers and their further (bio) chemical polymerization. The direct production of biopolymers can be achieved by microorganisms (polyhydroxyalkanoates, PHA), by algae (alginate etc.), by superior plants (pectin etc.) or by several types of producers, e.g. cellulose is produced by superior plants but also by bacteria, chitosan is produced by crustacean but also by fungi.

In principle, porous materials based on bio-based polymers are known from the state of the art, for example based on polysaccharides, polypeptides, polyphenolics such as cellulose, gelatine and lignin or mixtures of bio-based polymers. Preparation processes for lignin-based aerogels are also known from the state of the art.

For the purposes of the present invention, an aerogel is a porous material which has been produced by a sol-gel process in which the liquid phase has been removed from the gel under supercritical conditions.

Suitable aerogels preferably have a low density, and preferably high specific surface area, for example in the range of from 200 to 800 m²/g. Furthermore, a pore volume in the range of from 2.1 to 9.5 cm³/g for pore sizes <100 nm can preferably be obtained.

Preferably, the aerogel powder shows less than 60% shrinkage when exposed to humidity (60% rH, 48 h, 30 °C) as dry aerogel and less than 50% shrinkage when being loaded in the shape of a round bead with an average diameter of 3 mm.

The porous material according to the invention preferably has a density in the range of 0.005 to 1 g/cm³, preferably from 0.01 to 0.5 g/cm³ (determined according to DIN 53420).

The average pore diameter is determined by scanning electron microscopy and subsequent image analysis using a statistically significant number of pores. Corresponding methods are known to those skilled in the art. For characterization of the porous structure of aerogels a Nova 3000 Surface Area Analyzer from Quantachrome Instruments was used. It uses adsorption and desorption of nitrogen at a constant temperature of 77 K.

The aerogel powder used according to the invention preferably has a porosity of at least 70% by volume, in particular from 70 to 99% by volume, particularly preferably at least 80% by volume, very particularly preferably at least 85% by volume, in particular from 85 to 95% by volume. The porosity in % by volume means that the specified proportion of the total volume of the porous material comprises pores.

The aerogel used according to the present invention is based on at least one biopolymer. According to the present invention, preferably aerogels based on polyionic biopolymers may be used or aerogels based on mixtures of a biopolymer and further components. The aerogel may also comprise further components such as further biopolymers. Also, suitable further components known for the preparation of aerogels may be present. Suitable biopolymers and polyionic biopolymers are in principle known from the state of the art. Preferably, polyanionic biopolymers are used as polyionic biopolymers. Suitable ionic biopolymers are for example polysaccharides, in particular polysaccharides with carboxylic acid groups.

Suitable polyanionic biopolymers may be selected from the group consisting of alginates, pectin, modified cellulose, xanthan, carrageenan, hyaluronic acid.

According to a further embodiment, the present invention is also directed to the composition as disclosed above, wherein the polyionic biopolymer is a polyanionic biopolymer, preferably a polyionic biopolymer selected from the group consisting of alginates, pectin, modified cellulose, xanthan, carrageenan, hyaluronic acid.

Suitable aerogels based on polyionic biopolymers may be prepared using polyvalent metal ions which act as cross-linking metal ions. Such polyvalent metal ions include, for example, alkaline earth metal ions and transition metal ions which form poorly soluble compounds with the biopolymers.

Divalent or trivalent or tetravalent metal ions may for example be used. Trivalent metal ions such as aluminum or iron are particularly suitable. In addition, e.g. calcium, beryllium, barium, strontium, zinc, cobalt, nickel, copper, manganese, iron, chromium, vanadium, titanium, zirconium, cadmium, molybdenum, tungsten, ruthenium, rhodium, iridium, palladium, platinum, aluminum can also be used. According to the present invention, also mixtures of two or more polyvalent ions may be used, for example mixtures comprising divalent and trivalent ions.

Preferably, the polyvalent metal ion is a trivalent or tetravalent metal ion selected from the group consisting of aluminum, iron, zirconium.

According to a further embodiment, the present invention is also directed to the composition as disclosed above, wherein where the at least one polyionic biopolymer is gelled in water with at least one trivalent or tetravalent metal cation.

According to a further embodiment, the present invention is also directed to the composition as disclosed above, wherein the trivalent or tetravalent cation is selected from the group consisting of aluminum, iron, zirconium, tin, lead, manganese, molybdenum, tungsten, silicon, titanium.

According to the present invention, also aerogels based on biopolymers, and inorganic precursors, and polysaccharides with carboxylic acid groups may be used. Suitable biopolymers, and inorganic precursors, and polysaccharides with carboxylic acid groups are in principle known from the state of the art. Suitable are in particular water-soluble bio-polymers or bio-polymers which form swollen dispersions in water. Suitable polysaccharides with carboxylic acid groups are for example alginates, pectin, modified cellulose, xanthan, hyaluronic acid or modified starch. The use of these biopolymers and polyionic biopolymers such as polysaccharides and their derivatives are especially attractive because of their stability, availability, renewability and low toxicity.

According to the present invention, also mixtures of two or more biopolymers or mixtures of one or more biopolymers and a metal oxide may be used for the preparation of the aerogels used in the composition according to the present invention. For example mixtures comprising one or more polymers selected from the group consisting of biopolymers such as lignin and tannin, cellulose, bacterial cellulose, modified cellulose, starch, sugars, chitosan, polyhydroxyalkanoates, whey protein isolate, potato protein isolate, starch protein isolate, yeast protein, gelatin, collagen, casein or derivatives thereof or mixtures comprising an inorganic precursor and one or more polymers selected from the group consisting of bio-based phenolic polymers such as lignin and tannin, cellulose, bacterial cellulose, modified cellulose, starch, sugars, chitosan, polyhydroxyalkanoates, whey protein isolate, potato protein isolate, starch protein isolate, gelatin, collagen, casein or derivatives thereof or pea protein or yeast protein can be used.

The aerogels can also comprise further salts, in particular such salts that do not form gels, and customary auxiliaries known to those skilled in the art as further constituents. Suitable are for example substances which are water soluble or can be dispersed in water.

The aerogel may contain secondary materials (SM). Suitable substances used as secondary materials may for example be selected from hyaluronic acid, collagen, keratin, silk fibroin, tannin, lignin (both as anti-oxidant or sun protection factor), enzymes, polyvinylpyrrolidone (PVP), and povidone. Furthermore, suitable additives may be whitening actives; free radical scavengers, UV absorbers, barrier lipids, desquamatory actives, retinoids, tanning actives, skin brighteners, skin activators, chelators, flavonoids, moisturizing actives, exfoliating agents, anti-acne actives, anti-caking agents, anti-cellulite agents, anti-foaming agents, anti-fungal actives, anti-inflammatory actives, anti-microbial actives, anti-oxidants, antiperspirant/deodorant actives, anti-skin atrophy actives, anti-viral agents, anti-wrinkle actives, artificial tanning agents and accelerators, astringents, barrier repair agents, binders, buffering agents, bulking agents, chelating agents, colorants, dyes, enzymes, essential oils, film formers, flavors, humectants, hydrocolloids, light diffusers, nail enamels, opacifying agents, optical brighteners, optical modifiers, particulates, perfumes, pH adjusters, preservatives, sequestering agents, skin conditioners/moisturizers, skin feel modifiers, skin protectants, skin sensates, skin treating agents, skin exfoliating agents, skin lightening agents, skin soothing and/or healing agents, skin thickeners, sunscreen actives, topical anesthetics, vitamin compounds, and combinations thereof.

According to a further embodiment, the present invention is also directed to the composition as disclosed above, wherein the aerogel particles contain secondary materials (SM) selected from the group of lignin, tannin, polysaccharides, proteins, synthetic polymers including block copolymers, structuring polymers, synthetic polymers including block copolymers or semi-crystalline polymers, pigments, opacifiers, soft-focus fillers, spherical or lamellar inorganic or organic powders, perlite, sun protection agents, or inorganic precursors, colloidal silica, silicates, aluminates.

According to a further embodiment, the present invention is also directed to the composition as disclosed above, wherein the aerogel is a hybrid aerogel comprising
(A) 20-80% secondary materials (SM) by mass and 20% of ionically crosslinkable polycarboxylate polymer but at least 25 kg/m³ ionically crosslinkable polycarboxylate polymer, or
(B) 20-90% secondary materials (SM) by mass and 10% alginate by mass but at least 15 kg/m³ alginate, or
(C) 20-85% secondary materials (SM) by mass and 15% pectin by mass but at least 20 kg/m³ pectin, or
(D) 20-80% secondary materials (SM) by mass and 20% CMC by mass but at least 25 kg/m³ CMC, or
(E) 20-85% secondary materials (SM) by mass and 7.5% alginate by mass, but at least 15 kg/m³ alginate and at least 10 kg/m³ of non-alginate ionically crosslinkable polycarboxylate polymer.

It has been found that advantageous properties of the compositions are obtained when the polarity of the aerogel is in a similar range as the polarity of the non-aqueous phase. The polarity of the aerogel may for example be adjusted by the starting materials used or the ratio thereof or by the cation used for gelation. The polarity of the aerogel may also be adjusted by further treatment steps of the aerogel as such. Suitable treatments such as for example hydrophobization steps are in principle known.

The ratio of the non-aqueous phase and the aerogel used may vary in broad ranges as long as a composition is formed.

According to a further embodiment, the present invention is also directed to the composition as disclosed above, wherein the aerogel powder in the non-aqueous phase is present in an amount of the aerogel particle bulk density as lower limit to the aerogel bead density as upper limit relative to the density of the non-aqueous phase including the aerogel powder.

The aerogel present in the composition is used as a powder. The aerogel powder may be prepared using suitable processes. It may for example be prepared as a powder having a suitable particle size or the aerogel powder may be prepared from larger particles by milling, for example by milling aerogel beads.

According to a further embodiment, the present invention is also directed to the composition as disclosed above, wherein the aerogel powder is obtained by milling of aerogel beads of particle diameter in the range of 0.1 to 4 mm. The aerogel beads may be subjected to a suitable treatment before milling. According to a further embodiment, the present invention is also directed to the composition as disclosed above, wherein the aerogel optionally is treated at least at 130 °C for at least 2 hours to improve millability.

Suitable are in particular aerogels which have a similar or lower bulk density of the powder than of the aerogel beads. According to a further embodiment, the present invention is therefore directed to the composition as disclosed above, wherein the bulk density of the aerogel powder is equal or more than 5% lower than the bulk density of the aerogel beads, preferably more than 10%, in particular more than 15%.

Suitable processes for preparing aerogels based on biopolymers are in principle known to the person skilled in the art. Typically, the aerogels are obtained by processes including formation of a gel, solvent exchange and drying. According to a further embodiment, the present invention is also directed to the composition as disclosed above, wherein the aerogel beads are obtained by solvent exchange and supercritical drying or freeze drying.

It has been surprisingly found that the aerogels based on biopolymers can be used for the preparation of the compositions according to the present invention and already small amounts of the aerogel powder are sufficient to increase the viscosity of the resulting mixture with the non-aqueous phase. According to a further embodiment, the present invention is also directed to the composition as disclosed above, wherein the aerogel powder leads to a viscosity increase of the at least one non-aqueous phase.

The composition according to the present invention comprises at least one non-aqueous phase and an aerogel powder added to the at least one non-aqueous phase based on at least one polyionic biopolymer. The aerogel powder has a density of 30 g/l or less and the particle size of the aerogel powder is in the range of 1 to 250 micrometers.

The composition may contain further substances or the aerogel may be loaded with further additives. In the context of the present invention, it is also possible that the non-aqueous liquid or pasty phase comprises suitable additives.

Suitable additives may be triglycerides, vegetable oils, vegetable oil derivatives, acetoglyceride esters, alkyl esters, allcenyl esters, lanolin and its derivatives, wax esters, beeswax derivatives, sterols and phospholipids, and combinations thereof; hydrocarbon oils and waxes or silicone oils, and combinations thereof.

Suitable additives may also be pharmaceutical compositions containing one or more pharmaceutical agents preferably for oral administration. The one or more pharmaceutical agents are selected from therapeutic agents and diagnostic agents. Examples of suitable therapeutic agents include, but are not limited to, drugs acting at synaptic sites and neuroeffector junctional sites; general and local analgetics; hypnotics and sedatives; drugs for the treatment of psychiatric disorders such as depression and schizophrenia; anti-epileptics and anticonvulsants; drugs for the treatment of Parkinson's and Huntington's disease, aging and Alzheimer's disease; excitatory amino acid antagonists, neurotrophic factors and neuroregenerative agents; trophic factors; drugs aimed at the treatment of CNS trauma or stroke; drugs for the treatment of addiction and drug abuse; anti-obesity drugs; antacoids and anti-inflammatory drugs; chemotherapeutic agents for parasitic infections and diseases caused by microbes; immunosuppressive agents and anti-cancer drugs; hormones and hormone antagonists; heavy metals and heavy metal antagonists; antagonists for non-metallic toxic agents; cytostatic agents for the treatment of cancer; diagnostic substances for use in nuclear medicine; immunoactive and immunoreactive agents; transmitters and their respective receptor agonists and receptor antagonists, their respective precursors and metabolites; transporter inhibitors; antibiotics; antispasmodics; antihistamines; antinauseants; relaxants; stimulants; sense and antisense oligonucleotides; cerebral dilators; psychotropics; antimanics; vascular dilators and constrictors; anti-hypertensives; drugs for migraine treatment; hypnotics, hyperglycemic and hypoglycemic agents; anti-asthmatics; antiviral agents, preferably anti HIV agents; genetic material suitable for the DNA, si-RNA or antisense treatment of diseases; and mixtures thereof. Examples of suitable diagnostic agents include, but are not limited to, diagnostics useful in the diagnosis in nuclear medicine and in radiation therapy.

Suitable additives may also be fragrances or fragrance compositions which typically at least partially remain in the gel or the porous material under the conditions of the process according to the present invention. The fragrance may be any fragrant substance or mixture of substances, including natural and synthetic substances that provide a favorable aroma. In addition, the fragrance may contain auxiliary materials such as fixatives, extenders, stabilizers, and solvents. Examples of suitable fragrances include, but are not limited to, silicon oils, essential oils, absolutes, resinoids, resins, and synthetic perfume components such as hydrocarbons, alcohols, aldehydes, ketones, ethers, acids, esters, acetals, ketals, nitrites, including saturated and unsaturated compounds, aliphatic, carbocyclic and heterocyclic compounds. It should be recognized that certain fragrances may include additional components that function as, for example, carriers, diluents, stabilizers, etc. Exemplary additional components include glycols and vegetable oils. The reference to the fragrance component includes the fragrance as well as any additional component combined with the fragrance to provide a beneficial property such as stability, viscosity, etc. Examples of suitable fragrances, or perfumes, are for example provided in U.S. Pat. No. 5,234,610.

The composition according to the present invention is particularly suitable for personal care applications such as for example cosmetic applications. According to a further embodiment, the present invention is also directed to the composition as disclosed above, wherein the composition is a cosmetic composition, preferably a skin makeup, a sunscreen, a lipstick, a makeup removal, a deodorant, or a skin moisturizer composition.

According to a further aspect, the present invention also is directed to a method for preparing a composition, comprising:
(A) mixing non-aqueous ingredients comprising at least one non-aqueous liquid or pasty component, and at least one aerogel powder based on at least one polyionic biopolymer,
   where the viscosity of the composition is greater than the viscosity of the at least one non-aqueous phase, and
   wherein the aerogel powder has a density of 30 g/l or less and a particle size in the range of 0.5 to 250 micrometers.

The process according to the present invention comprises step (a) but may also comprise further steps. Preferably, in step (A), the composition is formed by gelling the non-aqueous component using the aerogel powder as viscosity modifying agent or as a gelling agent.

It is also possible to combine step (A) with further mixing steps. For example, the phases may be precombined to form a premix, and it is this premix which is placed in contact with the other component.

The components may be prepared separately and then mixed together without heating, without requiring the necessary presence of surfactants in order to achieve the formation of the composition. Thus, in addition to the advantages mentioned above, the claimed compositions may be readily prepared in a simple manner.

Advantageously, the mixing may be performed at room temperature. However, the process of the invention may comprise, if necessary, a step of heating the mixture, for example to improve the mixing.

It has surprisingly been found that the aerogel powders based on biopolymers may be used to increase the viscosity of the non-aqueous phases to obtain the composition according to the present invention which in turn has a viscosity which is suitable for the respective application. The original raw materials from which these aerogel powders are made do not present viscosity modifying or the high wet point properties compared to the corresponding aerogel powders. According to a further embodiment, the present invention is also directed to the method for preparing a composition as disclosed above, wherein the aerogel powder leads to a viscosity increase, i.e. the viscosity of the composition obtained is higher than the viscosity of the at least one non-aqueous phase.

The aerogel powder may be prepared in any suitable manner. Advantageously, the aerogel powder is prepared from aerogel beads, in particular aerogel beads of particle diameter in the range of 0.1 to 4 mm. The power may be prepared from the beads using any suitable method, for example milling using a suitable apparatus. Suitable methods are known to the person skilled in the art. Also, suitable treatment steps may be carried out to improve the millability, such as for example thermal treatment steps.

According to a further embodiment, the present invention is also directed to the method for preparing a composition as disclosed above, wherein the aerogel optionally is treated at least at 130 °C for at least 2 hours to improve millability.

According to a further embodiment, the present invention is also directed to the method for preparing a composition as disclosed above, wherein the aerogel powder is obtained by milling of aerogel beads of particle diameter in the range of 0.1 to 4 mm.

It has been found that in particular aerogels gelled with trivalent or tetravalent cations have the advantage that the bulk density of the aerogel powder is equal or lower than the bulk density of the aerogel beads.

According to a further embodiment, the present invention is also directed to the method for preparing a composition as disclosed above, wherein the bulk density of the aerogel powder is equal or more than 5% lower than the bulk density of the aerogel beads.

Suitable processes for preparing aerogel powder or aerogel beads are in principle known to the person skilled in the art.

Suitable processes may for example comprise the preparation of a gel, solvent exchange and drying steps.

According to a further embodiment, the present invention is also directed to the method for preparing a composition as disclosed above, wherein where the aerogel beads are obtained by solvent exchange and supercritical drying or freeze drying.

Suitable aerogels might for example be prepared by a process at least comprising the steps of:
a) providing a mixture (M1) comprising optionally one compound (C1) selected from the group consisting of biopolymers and at least one polyionic biopolymer as component (C2) and water,
b) bringing mixture (M1) into contact with an aqueous solution of a polyvalent metal ion to prepare a gel (A)
c) exposing the gel (A) obtained in step b) to a water miscible solvent (L) to obtain a gel (B)
d) drying of the gel (B) obtained in step c).

Suitable aerogels might also be prepared by a process at least comprising the steps of:
a) providing a mixture (M1) comprising at least one compound (C1) selected from water-soluble biopolymers and inorganic precursors, and at least one water-soluble polysaccharide with carboxylic acid groups as component (C2) and water,
b) bringing mixture (M1) into contact with an aqueous solution of a polyvalent metal ion to prepare a gel (A)
c) exposing the gel (A) obtained in step b) to a water miscible solvent (L) to obtain a gel (B),
d) drying of the gel (B) obtained in step c).

Suitable inorganic precursors in the context of the present invention have to be soluble or at least partially soluble in the mixture (M1) and have to solidify in the gelation step.

For the purposes of the present invention, a gel is a crosslinked system based on a polymer which is present in contact with a liquid (known as solvogel or lyogel), or with water as liquid (aquagel or hydrogel). Here, the polymer phase forms a continuous three-dimensional network.

In the context of the present invention, water-soluble means that the solubility in water is sufficient to form a solution which can be used for preparing a gel. In the context of the present invention, also aqueous swollen dispersions may be used for the preparation of a gel.

According to the present invention, a gel is formed from the components of mixture (M1) and at least one polyvalent metal ion. The components (C1) and (C2) used for the process of the present invention have to be suitable to allow the formation of a gel with the polyvalent metal ion, in particular have to have suitable functional groups.

Properties of the aerogels can be customized by adjusting the composition of mixture (M1), the reaction conditions at the stage of the formation of the hydrogel (gel (A)), or during solvent exchange as well as in the drying step. According to the present invention, it is possible to influence the properties of the hydrogels and/or aerogels by varying the ratio of the components, as well as by controlling the parameters of step b) and also by introducing a wide range of organic and inorganic materials in the gel matrix.

According to step b) of the present invention, mixture (M1) is brought into contact with an aqueous solution of a polyvalent metal ion to prepare a gel (A). Suitable mixing steps are in principle known to the person skilled in the art. It is for example possible to add mixture (M1) dropwise to the aqueous solution of the polyvalent metal ion, in particular for the preparation of aerogel beads. It is also possible that mixture (M1) is provided in the pores of a carrier material or in admixture with fibers before bringing it into contact with the aqueous solution of a polyvalent metal ion to prepare a gel (A). Also, mixture (M1) can be brought into contact with the polyvalent metal ion in an emulsion or in a spray process.

Gelling is known per se to a person skilled in the art and is described, for example, in WO 2009/027310 on page 21, line 19 to page 23, line 13.

Preferably, the conditions are adjusted and the hydrogel, alcogel and/or aerogel exhibit a round shape. Preferably, according to step b), a round bead with an average diameter in the range of from 0.5 to 3 mm is obtained. Preferably, no crosslinking or hydrophobization via covalent chemical reaction occurs.

Preferably, temperature and pressure in step b) are adjusted to conditions under which a gel is formed. A suitable temperature might be in the range of from 5 to 60 °C, preferable in the range of from 15 to 35 °C. According to a further embodiment, the present invention also relates to a process as described above, wherein step b) is carried out at a temperature in the range of from 5 to 60 °C.

The rate of formation of the insoluble gel can be controlled very exactly and easily by choosing suitable conditions for step b).

Gel (A) obtained in step b) is a gel comprising water, i.e. a hydrogel. According to the present invention gel (A) obtained in step b) is exposed to a water-miscible solvent (L) to obtain a gel (B) in step c) of the process of the present invention.

According to the present invention, a water-miscible solvent (L) is used in step c). In the context of the present invention, water-miscible means that the solvent is at least partially miscible with water in order to allow an exchange of solvent in the gel.

Solvent exchange is carried out either by soaking the gel directly in the new solvent (one-step) or by following a sequential soaking (multi-step) in different water-to-new solvent mixtures with increasing content in the new solvent after a certain time (exchange frequency) in the previous soaking step (Robitzer et al., 2008, Langmuir, 24 (21), 12547-12552). The solvent chosen for water replacement must satisfy the requirements of not dissolving the gel structure, being completely soluble with the solvent which precedes them (water) and preferably also accepted for manufacturing of pharmaceuticals. Furthermore, in case the process encompasses a step of supercritical drying, solvent (L) preferably is at least partially miscible with the supercritical medium.

The solvent (L) can in principle be any suitable compound or mixture of a plurality of compounds, which meets the above requirements with the solvent (L) being liquid under the temperature and pressure conditions of step c).

Possible solvents (L) are, for example, alcohols, ketones, aldehydes, alkyl alkanoates, organic carbonates, amides such as formamide and N-methylpyrollidone, sulfoxides such as dimethyl sulfoxide, aliphatic and cycloaliphatic halogenated or non-halogenated hydrocarbons, halogenated or non-halogenated aromatic compounds and fluorine-containing ethers. Mixtures of two or more of the abovementioned compounds are likewise possible.

In many cases, particularly suitable solvents (L) are obtained by using two or more completely miscible compounds selected from the above mentioned solvents.

Suitable solvents are in particular alcohols and ketones, for example C1 to C6 alcohols and C1 to C6 ketones and mixtures thereof.

According to a further embodiment, the present invention also relates to a process as described above, wherein the solvent (L) used in step c) is selected from the group consisting of C1 to C6 alcohols and C1 to C6 ketones and mixtures thereof.

Particularly suitable are alcohols such as methanol, ethanol and isopropanol and ketones such as acetone, and methyl ethyl ketone.

The solvent exchange according to step b) might be carried out in one step, 2 steps, 3 steps or in multiple steps with varying concentration of the solvent. According to a preferred embodiment, gels (A) are successively immersed in ethanol/water mixtures with concentrations of for example 30, 60, 90 and 100 wt % for 5 min to 12 h in each depending on the particle size and porosity.

In step c), gel (B) is obtained. According to step d) of the process of the present invention, gel (B) obtained in step c) is dried.

Drying in step (d) takes place in a known manner. Drying under supercritical conditions is preferred, preferably after replacement of the solvent by CO₂ or other solvents suitable for the purposes of supercritical drying. Such drying is known per se to a person skilled in the art. Supercritical conditions characterize a temperature and a pressure at which CO₂ or any solvent used for removal of the gelation solvent is present in the supercritical state. In this way, shrinkage of the gel body on removal of the solvent can be reduced.

It is also possible to dry the gels obtained by conversion of the liquid comprised in the gel into the gaseous state at a temperature and a pressure below the critical temperature and the critical pressure of the liquid comprised in the gel.

Preferably, the drying of the gel obtained is preferably carried out by converting the solvent (L) into the gaseous state at a temperature and a pressure below the critical temperature and the critical pressure of the solvent (L). Accordingly, drying is preferably carried out by removing the solvent (L) which was present in the reaction without prior replacement by a further solvent.

Such methods are likewise known to those skilled in the art and are described in WO 2009/027310 on page 26, line 22 to page 28, line 36.

Drying according to step d) may be carried out by converting the liquid comprised in the gel into the gaseous state at a temperature and a pressure below the critical temperature and the critical pressure of the liquid comprised in the gel. It is also possible that the drying according to step d) is carried out under supercritical conditions.

Preferably, the aerogel is prepared in the form of beads, for example having an average diameter in the range of 0.5 mm to 3 mm and the powder is obtained by milling.

According to the present invention, for example cosmetic or medical active substances or pharmaceutical agents or agents for agriculture or actives for food or fragrances or fragrance compositions may be introduced during the preparation process as set out above which are then at least partially present in the aerogel powder and may also be released under suitable conditions.

According to the present invention it is also possible to bring the aerogel powder into contact with a suitable liquid composition comprising cosmetic or medical active substances or pharmaceutical agents or agents for agriculture or actives for food or fragrances or fragrance compositions which are then at least partially adsorbed. Preferably, the process comprises mixing the porous material with the liquid composition and allowing a sufficient time for an effective amount of said composition to be adsorbed onto and/or absorbed by the porous material. The liquid composition may be a solution, preferably a highly concentrated solution. Suitable are for example solutions or dispersions comprising cosmetic or medical active substances or pharmaceutical agents or agents for agriculture or actives for food or fragrances or fragrance compositions and a suitable solvent. In the context of the present invention, it is also possible to use liquid active substances as such without the addition of a solvent. The porous material containing the adsorbed substances may then be separated from any remaining solution or dispersion. It is also possible to release the adsorbed substances under suitable conditions according to the present invention.

According to a further aspect, the present invention relates to the use of the composition as disclosed above or a composition obtained or obtainable by the process as disclosed above for food applications, for cosmetic applications, for biomedical applications, for agricultural applications, for consumer applications, for coating applications, for paint applications, for construction applications, for adhesive applications, for fragrance release applications, or for pharmaceutical applications. Such cosmetic applications include for example products for facial treatment or as skin scrubbing or cleansing or protective products such as products for UV protection or products including antioxidants.

Preferred embodiments may be found in the claims and the description. Combinations of preferred embodiments do not go outside the scope of the present invention. Preferred embodiments of the components used are described below.

The present invention is further illustrated by the following set of embodiments and combinations of embodiments resulting from the dependencies and back-references as indicated. In particular, it is noted that in each instance where a range of embodiments is mentioned, for example in the context of a term such as “The composition of any one of embodiments 1 to 4”, every embodiment in this range is meant to be explicitly disclosed for the skilled person, i.e. the wording of this term is to be understood by the skilled person as being synonymous to “The composition of any one of embodiments 1, 2, 3 and 4”. Further, it is explicitly noted that the following set of embodiments represents a suitably structured part of the general description directed to preferred aspects of the present invention, and, thus, suitably supports, but does not represent the claims of the present invention.

1. A composition in the form of an organogel, emulsion or bigel, comprising
(i) at least one non-aqueous liquid or pasty phase
(ii) an aerogel powder added to the at least one non-aqueous phase based on at least one biopolymer, in particular at least one polyionic biopolymer,
where the viscosity of the composition is greater than the viscosity of the at least one non-aqueous phase, and
wherein the aerogel powder has a density of 30 g/l or less and where the particle size of the aerogel powder is in the range of 0.5 to 250 micrometers.

2. The composition according to embodiment 1, wherein where the at least one polyionic biopolymer is gelled in water with at least one trivalent or tetravalent metal cation.

3. The composition according to embodiment 2, wherein the trivalent or tetravalent cation is selected from the group consisting of aluminum, iron, zirconium, tin, lead, manganese, molybdenum, tungsten, silicon, titanium.

4. The composition according to any one of embodiments 1 to 3, wherein the polyionic biopolymer is a polyanionic biopolymer, preferably a polyionic biopolymer selected from the group consisting of alginates, pectin, modified cellulose, xanthan, carrageenan, hyaluronic acid.

The composite according to any one of embodiments 1 to 4, wherein the aerogel particles contain secondary materials (SM) selected from the group of lignin, tannin, polysaccharides, proteins, synthetic polymers including block copolymers, structuring polymers, synthetic polymers including block copolymers or semi-crystalline polymers, pigments, opacifiers, soft-focus fillers, spherical or lamellar inorganic or organic powders, perlite, sun protection agents, or inorganic precursors.

6. The composite according to any one of embodiments 1 to 5, wherein the aerogel is a hybrid aerogel comprising
(A) 20-80% secondary materials (SM) by mass and 20% of ionically crosslinkable polycarboxylate polymer but at least 25 kg/m³ ionically crosslinkable polycarboxylate polymer, or
(B) 20-90% secondary materials (SM) by mass and 10% alginate by mass but at least 15 kg/m³ alginate, or
(C) 20-85% secondary materials (SM) by mass and 15% pectin by mass but at least 20 kg/m³ pectin, or
(D) 20-80% secondary materials (SM) by mass and 20% CMC by mass but at least 25 kg/m³ CMC, or
(E) 20-85% secondary materials (SM) by mass and 7.5% alginate by mass, but at least 15 kg/m³ alginate and at least 10 kg/m³ of non-alginate ionically crosslinkable polycarboxylate polymer.

7 The composition according to any one of embodiments 1 to 6, wherein the non-aqueous phase contains one or more of esters of saturated or unsaturated, linear or branched C1-C26 aliphatic monoacid or polyacids and of saturated or unsaturated, linear or branched C1-C26 aliphatic monoalcohol or polyalcohol, or silicones, or silicon oils or fluorosilicon oils, non-aqueous fragrances, aromas, nutrients, preservatives, pH regulators, film formers, dextran esters, sucrose esters, moisturizers, synthetic polymers including block copolymers or semi-crystalline polymers, structuring polymers, sun protection agents, silicone elastomers, pigments, mixed pigments or pigment composites, opacifiers, blurring fillers, soft-focus fillers, spherical or lamellar inorganic or organic powders, perlite, sun protection agents.

8. The composition according to any one of embodiments 1 to 7, wherein the aerogel powder in the non-aqueous phase is present in an amount of the aerogel particle bulk density as lower limit to the aerogel bead density as upper limit relative to the density of the non-aqueous phase including the aerogel powder.

9. The composition according to any one of embodiments 1 to 8, wherein the aerogel powder is obtained by milling of aerogel beads of particle diameter in the range of 0.1 to 4 mm.

10. The composition according to any one of embodiments 1 to 9, wherein the bulk density of the aerogel powder is equal or more than 5% lower than the bulk density of the aerogel beads.

11. The composition according to any one of embodiments 1 to 10, wherein the aerogel beads are obtained by solvent exchange and supercritical drying or freeze drying.

12. The composition according to any one of embodiments 1 to 11, wherein the aerogel powder leads to a viscosity increase of at least 100% of the at least one non-aqueous phase.

13. The composition according to any one of embodiments 1 to 12, wherein the aerogel is treated at least at 130 °C for at least 2 hours to improve millability.

14. The composition according to any one of embodiments 1 to 13, wherein the composition is a cosmetic composition, preferably a skin makeup, a sunscreen, a lipstick, a makeup removal, or a skin moisturizer composition.

15. A method for preparing a composition, comprising:
(A) mixing non-aqueous ingredients comprising at least one non-aqueous liquid or pasty component, and at least one aerogel powder based on at least one polyionic biopolymer,
wherein the aerogel powder has a density of 30 g/l or less and a particle size in the range of 0.5 to 250 micrometers, where the viscosity of the composition is greater than the viscosity of the at least one non-aqueous phase.

16. The method according to embodiment 15, wherein the aerogel powder is obtained by milling of aerogel beads of particle diameter in the range of 0.1 to 4 mm.

17. The method according to embodiment 15 or 16, wherein the bulk density of the aerogel powder is equal or more than 5% lower than the bulk density of the aerogel beads.

18. The method according to any one of embodiments 15 to 17, wherein where the aerogel beads are obtained by solvent exchange and supercritical drying or freeze drying.

19. The method according to any one of embodiments 15 to 18, wherein the aerogel powder leads to a viscosity increase of at least 100% of the at least one non-aqueous phase.

20. The method according to any one of embodiments 15 to 19, wherein the aerogel optionally is treated at least at 130 °C for at least 2 hours to improve millability.

21. The method according to any one of embodiments 15 to 20, wherein where the at least one biopolymer is gelled in water with at least one trivalent or tetravalent metal cation.

22. The method according to any one of embodiments 15 to 21, wherein the trivalent or tetravalent cation is selected from the group consisting of aluminum, iron, zirconium, tin, lead, manganese, molybdenum, tungsten, silicon, titanium.

23. The method according to any one of embodiments 15 to 22, wherein the polyionic biopolymer is a polyanionic biopolymer, preferably a polyionic biopolymer selected from the group consisting of alginates, pectin, modified cellulose, xanthan, carrageenan, hyaluronic acid.

24 The method according to any one of embodiments 15 to 23, wherein the aerogel particles contain secondary materials (SM) selected from the group of lignin, tannin, polysaccharides, proteins, synthetic polymers including block copolymers, structuring polymers, synthetic polymers including block copolymers or semi-crystalline polymers, pigments, opacifiers, soft-focus fillers, spherical or lamellar inorganic or organic powders, perlite, sun protection agents, or inorganic precursors.

25. The method according to any one of embodiments 15 to 24, wherein the aerogel is a hybrid aerogel comprising
(A) 20-80% secondary materials (SM) by mass and 20% of ionically crosslinkable polycarboxylate polymer but at least 25 kg/m³ ionically crosslinkable polycarboxylate polymer, or
(B) 20-90% secondary materials (SM) by mass and 10% alginate by mass but at least 15 kg/m³ alginate, or
(C) 20-85% secondary materials (SM) by mass and 15% pectin by mass but at least 20 kg/m³ pectin, or
(D) 20-80% secondary materials (SM) by mass and 20% CMC by mass but at least 25 kg/m³ CMC, or
(E) 20-85% secondary materials (SM) by mass and 7.5% alginate by mass, but at least 15 kg/m³ alginate and at least 10 kg/m³ of non-alginate ionically crosslinkable polycarboxylate polymer.

26. The method according to any one of embodiments 15 to 25, wherein the non-aqueous phase contains one or more of esters of saturated or unsaturated, linear or branched C1-C26 aliphatic monoacid or polyacids and of saturated or unsaturated, linear or branched C1-C26 aliphatic monoalcohol or polyalcohol, or silicones, or silicon oils or fluorosilicon oils, non-aqueous fragrances, aromas, nutrients, preservatives, pH regulators, film formers, dextran esters, sucrose esters, moisturizers, synthetic polymers including block copolymers or semi-crystalline polymers, structuring polymers, sun protection agents, silicone elastomers, pigments, mixed pigments or pigment composites, opacifiers, blurring fillers, soft-focus fillers, spherical or lamellar inorganic or organic powders, perlite, sun protection agents.

27. The method according to any one of embodiments 15 to 26, wherein the aerogel powder in the non-aqueous phase is present in an amount of the aerogel particle bulk density as lower limit to the aerogel bead density as upper limit relative to the density of the non-aqueous phase including the aerogel powder.

28. The use of a composition according to any of embodiments 1 to 14 or a composition obtained or obtainable by the process according to any of embodiments 15 to 20 for food applications, for cosmetic applications, for biomedical applications, for agricultural applications, for consumer applications, for construction applications, for adhesive applications, for coating applications, for paint applications, for fragrance release applications, or for pharmaceutical applications.

Examples will be used below to illustrate the invention.

### EXAMPLES

### Aerogel Preparation

Aerogel particles were produced as follows: Carboxymethyl cellulose (CMC, TEXTURECEL 100 G from IFF), sodium alginate (SA, Protanal LF120 from IFF) and colloidal silica (CS, Levasil CS15-340P from Nouryon) were blended at a given target concentration at different ratios with water to obtain a precursor solution of 1 l volume.

The precursor solution was dripped into a 5 l salt bath to form hydrogel particles. Hydrogel particle size was controlled by the dripping method between 1 mm and 2 mm diameter. The water in the hydrogel particles was exchanged to ~98% ethanol over ten steps against 98% ethanol by immersion in ten times of the particle volume of 98% ethanol for ~3 hours. The obtained alcogel particles were dried with supercritical CO₂ to obtain aerogel particles. In some cases the aerogel particles were hydrophobized by placing 1 l particles in a holder above 30 ml hexamethyldisiloxane in a glass jar in a closed 3 l autoclave at 130 °C for 12 h. The aerogel particles were milled to powder (particle size 10-250 μm) by blending in a kitchen blender for ~60 s.
Bioaerogel 1: Polycarboxylate/CS 80:20 with polycarboxylate 75:25 CMC/SA, 2.4 wt %, calcium chloride/aluminum chloride gelation bath (20 g/l CaCl₂·2H₂O, 20 g/l AlCl₃·6H₂O), milled density 20 g/l.
Bioaerogel 2: Polycarboxylate/CS silica 80:20 with polycarboxylate 75:25 CMC/SA, 2.4 wt %, calcium chloride/aluminum chloride gelation bath (20 g/l CaCl₂·2H₂O, 20 g/l AlCl₃·6H₂O), milled density 20 g/l, hydrophobized.
Bioaerogel 3: Polycarboxylate with 75:25 CMC/SA, 2.0 wt %, aluminum chloride gelation bath (30 g/l AlCl₃·6H₂O), milled density 15 g/l.

### Viscosity Measurements

For viscosity measurements, 100 g of a liquid phase was mixed with aerogel powder in different quantities, stirred briefly with a spatula and homogenized with a high-speed homogenizer (Sil-verson L5M-A) at 8000 rpm for 30 s. The viscosity was measured in a IKA rotavisc me-vi rotary viscosimeter with 150 ml mixture in a glass beaker at 50 rpm for 30 s.

### Absorption Capacity at Wet Point

For measurement of the absorption capacity at the wet point, the procedure described in WP2012084780 was used using various liquid phases: The absorption capacity measured at the wet point, noted Wp, corresponds to the amount of oil that needs to be added to 100 g of particles in order to obtain a homogeneous paste. It is measured according to the wet point method or the method for determining the oil uptake of a powder described in standard NF T 30-022. It corresponds to the amount of oil adsorbed onto the available surface of the powder and/or absorbed by the powder by measuring the wet point, described below: An amount, m=2 g, of powder is placed on a glass plate, and the oil is then added dropwise. After addition of 4 to 5 drops of oil to the powder, mixing is performed using a spatula, and addition of oil is continued until conglomerates of oil and powder have formed. From this point, the oil is added one drop at a time and the mixture is then triturated with the spatula. The addition of oil is stopped when a firm, smooth paste is obtained. This paste must be able to be spread on the glass plate without cracking or forming lumps. The volume Vs (expressed in ml) of oil used is then noted. The oil uptake corresponds to the ratio Vs/m.

TABLE 1
Viscosity measurements.

| Bioaerogel 1 | | | | Bioaerogel 2 | | | | | | Bioaerogel 3 | |
|---|---|---|---|---|---|---|---|---|---|---|---|
| Ethanol | | Sunflower oil | | Ethanol | | Sunflower oil | | Dodecane | | Water | |
| wt % | Viscosity | wt % | Viscosity | wt % | Viscosity | wt % | Viscosity | wt % | Viscosity | wt % | Viscosity |
| aerogel | mPa*s | aerogel | mPa*s | aerogel | mPa*s | aerogel | mPa*s | aerogel | mPa*s | aerogel | mPa*s |
| 0.0% | 8 | 0.0% | 70 | 0.8% | 9 | 0.0% | 70 | 0.0% | 10 | 0.0% | 8 |
| 0.8% | 20 | 0.7% | 96 | 1.6% | 10 | 0.4% | 76 | 1.0% | 15 | 0.7% | 16 |
| 1.2% | 46 | 1.3% | 235 | 2.3% | 15 | 1.0% | 90 | 2.0% | 44 | 1.2% | 130 |
| 1.6% | 152 | 2.0% | 1132 | 2.9% | 22 | 2.0% | 234 | 2.7% | 152 | 1.6% | 700 |
| 1.9% | 345 | 2.3% | 2920 | 3.4% | 30 | 2.3% | 600 | 3.1% | 250 | | |
| 2.4% | 800 | | | 4.5% | 40 | | | 3.6% | 453 | | |
| | | | | 5.0% | 55 | | | 4.1% | 700 | | |
| | | | | 5.7% | 93 | | | | | | |

Bioaerogel 3

| | | | | | | | | Propylene | | Silicone oil | |
|---|---|---|---|---|---|---|---|---|---|---|---|
| 1,4-Butanediol | | Triethyl citrate | | Brij 30 | | PEG 200 | | carbonate | | M350 CST | |
| wt % | Viscosity | wt % | Viscosity | wt % | Viscosity | wt % | Viscosity | wt % | Viscosity | wt % | Viscosity |
| aerogel | mPa*s | aerogel | mPa*s | aerogel | mPa*s | aerogel | mPa*s | aerogel | mPa*s | aerogel | mPa*s |
| 0.0% | 86 | 0.0% | 46 | 0.0% | 50 | 0.0% | 66 | 0.0% | 16 | 0.0% | 22 |
| 0.5% | 134 | 0.3% | 58 | 0.6% | 88 | 0.3% | 82 | 0.7% | 32 | 0.8% | 64 |
| 1.1% | 305 | 0.8% | 150 | 1.1% | 245 | 0.8% | 234 | 1.4% | 750 | 1.5% | 180 |
| 1.4% | 825 | 1.3% | 500 | 1.4% | 700 | 1.3% | 950 | | | 2.0% | 644 |

TABLE 2
Measurements of liquid absorption capacity at wet point.

| | | Wet | Wet |
|---|---|---|---|
| | Density | point | point |
| | liquid | min | max |
| | g/ml | ml/g | ml/g |
| Cabot IC3110 silica aerogel + sun- | 0.92 | 14 | 17 |
| flower oil | | | |
| Aerosil A380 + sunflower oil | 0.92 | 14 | 16 |
| IFF Protanal LF120 sodium alginate | 0.92 | 1.1 | 1.6 |
| Dupont Texturecel 500 CMC | 0.92 | 1.6 | 2.2 |
| Bioaerogel 1 + ethanol | 0.79 | 35 | 42 |
| Bioaerogel 1 + HMDSO | 0.76 | 26 | 33 |
| Bioaerogel 1 + dodecane | 0.75 | 33 | 40 |
| Bioaerogel 2 + dodecane | 0.75 | 24 | 27 |
| Bioaerogel 3 + sunflower oil | 0.92 | 39 | 42 |
| Bioaerogel 3 + silicone oil M350 CST | 0.94 | 32 | 37 |

### LITERATURE CITED

“Aerogels as porous structures for food applications: Smart ingredients and novel packaging materials”, Lara Manzocco et al in Food Structure, vol 28, 2021, 100188
“Iron (iii)-cross-linked alginate hydrogels: a critical review”, Daniel Massana Roquero et al. Mater. Adv., 2022, 3, 1849-1873
PCT/EP2023/055870
WO 2009/027310
Robitzer et al., 2008, Langmuir, 24 (21), 12547-12552

1. A composition in the form of an organogel, emulsion or bigel, the composition comprising:
(i) at least one non-aqueous liquid or pasty phase
(ii) an aerogel powder based on at least one polyionic biopolymer,
wherein the viscosity of the composition is greater than the viscosity of the at least one non-aqueous liquid or pasty phase, and wherein the aerogel powder has a density of 30 g/l or less and wherein the particle size of the aerogel powder is in the range of 0.5 to 250 micrometers.

2. The composition according to claim 1, wherein the aerogel powder is based on at least one polyionic biopolymer gelled in water with at least one trivalent or tetravalent metal cation.

3. The composition according to claim 2, wherein the at least one trivalent or tetravalent metal cation is selected from the group consisting of aluminum, iron, zirconium, tin, lead, manganese, molybdenum, tungsten, silicon, and titanium.

4. The composition according to claim 1, wherein the at least one polyionic biopolymer is a polyanionic biopolymer.

5. The composition according to claim 1, wherein the aerogel particles contain secondary materials (SM) selected from the group consisting of lignin, tannin, polysaccharides, proteins, synthetic polymers including block copolymers, structuring polymers, synthetic polymers including block copolymers or semi-crystalline polymers, pigments, opacifiers, soft-focus fillers, spherical or lamellar inorganic or organic powders, perlite, sun protection agents, and inorganic precursors.

6. The composition according to claim 1, wherein the aerogel powder is a hybrid aerogel comprising
(A) 20-80% secondary materials (SM) by mass and 20% of ionically crosslinkable polycarboxylate polymer, but at least 25 kg/m³ ionically crosslinkable polycarboxylate polymer, or
(B) 20-90% secondary materials (SM) by mass and 10% alginate by mass, but at least 15 kg/m³ alginate, or
(C) 20-85% secondary materials (SM) by mass and 15% pectin by mass, but at least 20 kg/m³ pectin, or
(D) 20-80% secondary materials (SM) by mass and 20% CMC by mass, but at least 25 kg/m³ CMC, or
(E) 20-85% secondary materials (SM) by mass and 7.5% alginate by mass, but at least 15 kg/m³ alginate and at least 10 kg/m³ of non-alginate ionically crosslinkable polycarboxylate polymer.

7. The composition according to claim 1, wherein the non-aqueous liquid or pasty phase contains one or more esters selected from the group consisting of saturated or unsaturated, linear or branched C1-C26 aliphatic monoacid or polyacids and of saturated or unsaturated, linear or branched C1-C26 aliphatic monoalcohol or polyalcohol, or silicones, or silicon oils or fluorosilicon oils, non-aqueous fragrances, aromas, nutrients, preservatives, pH regulators, film formers, dextran esters, sucrose esters, moisturizers, synthetic polymers including block copolymers or semi-crystalline polymers, structuring polymers, sun protection agents, silicone elastomers, pigments, mixed pigments or pigment composites, opacifiers, blurring fillers, soft-focus fillers, spherical or lamellar inorganic or organic powders, perlite, and sun protection agents.

8. The composition according to claim 1, wherein the aerogel powder in the composition is present in an amount of the aerogel particle bulk density as lower limit to the aerogel bead density as upper limit relative to the density of the non-aqueous phase including the aerogel powder.

9. The composition according to claim 1, wherein the composition is a cosmetic composition.

10. A method for preparing a composition, the method comprising:
(A) mixing non-aqueous ingredients comprising at least one non-aqueous liquid or pasty component, and at least one aerogel powder based on at least one polyionic biopolymer,
wherein the at least one aerogel powder has a density of 30 g/l or less and a particle size in the range of 0.5 to 250 micrometers, wherein the composition is an organogel, emulsion or bigel and the viscosity of the composition is greater than the viscosity of the at least one non-aqueous liquid or pasty component.

11. The method according to claim 10, wherein the at least one aerogel powder is obtained by milling of aerogel beads of particle diameter in the range of 0.1 to 4 mm.

12. The method according to claim 11, wherein the bulk density of the at least one aerogel powder is equal or more than 5% lower than the bulk density of the aerogel beads.

13. The method according to claim 10, wherein the at least one aerogel powder leads to a viscosity increase of at least 100% of the at least one non-aqueous liquid or pasty component.

14. The method according to claim 10, wherein the at least one aerogel powder optionally is treated at least at 130 °C for at least 2 hours to improve millability.

15. The composition according to claim 1, wherein the composition is configured to be used for food applications, for cosmetic applications, for biomedical applications, for agricultural applications, for consumer applications, for construction applications, for adhesive applications, for coating applications, for paint applications, for fragrance release applications, or for pharmaceutical applications.
